(19) United States
(12) Patent Application Publication
(10) Pub. No.: US 2010/0255127 A1
(43) Pub. Date: Oct. 7, 2010

(54) AROMATASE INHIBITOR

(75) Inventors: Hisayoshi Norimoto, Toyama (JP); Yasuo Morimoto, Toyama (JP)

Correspondence Address: GRIFFIN & SZIPL, PC, SUITE PH-1, 2300 NINTH STREET, SOUTH, ARLINGTON, VA, 22204, US

(73) Assignee: KRACIE PHARMA, LTD., Tokyo, JP (JP)

(21) Appl. No.: 12/744,320

(22) PCT Filed: Nov. 20, 2008
(86) PCT No.: PCT/JP2008/071072

§ 371 Date: May 21, 2010

(30) Foreign Application Priority Data
Nov. 21, 2007 (JP) 2007-301111

(51) Int. Cl. A61K 36/00; A61K 36/49; A61K 36/736; A61K 36/15; A61K 36/38; A61K 36/82; A61P 3/00; A61P 15/00

(52) U.S. Cl. 424/729; 424/725; 424/771; 424/774; 424/769; 424/773; 424/770; 424/730

(57) ABSTRACT

Disclosed is a safe substance which can inhibit the activity of aromatase (an enzyme capable of converting androgen into estrogen) to thereby effectively treat and/or prevent a sex hormone-dependent disease such as breast cancer occurring in a female person after menopause, as well as a climacteric disorder in a male person and metabolic syndrome caused by the accumulation of a visceral fat. Specifically disclosed is a therapeutic and/or prophylactic agent for a sex hormone-dependent disease, which is characterized by comprising at least one herbal extract selected from the group consisting of “Kokeiten” (golden root), prunella spike, sweet hydrangea leaf, milk thistle, a jasmine tea, “Bokusoku” (a bark of Quercus acutissima or a closely related plant thereof), “Tencha” (sweet tea), “Karensou” (Eclipta prostrata), “Youbaihi” (a bark of Myrica rubra Sie b. et Zucc.), French maritime pine, betel palm, asparagus, “Rouro” (a root of Rhaponticum unifloru m DC. or Echinops latifolius Tausch), “Ryoukyou” (a rhizome of Alpinia officinarum Hance), rooibos tea, rhubarb, pu-erh tea, green tea, “Ougon” (a root of Scutellaria baicalensis Georgi), St. John's wort (Hypericum perforatum L.), licorice, “Senrikou” (Senecio scandens Buch.-Ham.), wintergreen, “Kashi” (a matured fruit of Terminalia chebula Retz.), “Yagotou” (a bark of Mallotus japonicus), polygnum root, barrenwort (Epimedium Herb), guarana, “Ouhi” (a bark of Prunus jamasakura Sieb. ex. Koidz. or a closely related plant thereof), Argy's wormwood, sticky rehmannia, Japanese cornel, Asiasarum root, cinnamon, peony root, pine needle and amla fruit.

This is a National Phase Application in the United States of International Patent Application No. PCT/JP2008/071072 filed Nov. 20, 2008, which claims priority on Japanese Patent Application No. 2007-031111, filed Nov. 21, 2007. The entire disclosures of the above patent applications are hereby incorporated by reference.

### TECHNICAL FIELD

The present invention relates to a medical agent having an aromatase-inhibitory activity. More specifically, the present invention relates to a drug effective for treatment and/or prevention of not only breast cancer of female after menopause, but also sex hormone-dependent diseases such as male menopausal disorders and metabolic syndrome due to accumulation of visceral fats, etc., by inhibiting activity of aromatase which is an enzyme converting a male hormones into a female hormones in a living body whereby limiting decrease in a male hormone or increase in a female hormone.

### BACKGROUND ART

It has been clarified in recent years that, in estrogen-dependent diseases such as hysteromyroma, endometriosis, endometrial cancer, breast cancer of female after menopause, etc., estrogen synthesis is accelerated at the local portion of the lesion (in situ estrogen), and the local estrogen generated thereby deeply participates in growth and evolution of the lesion (Non-Patent Literatures 1 and 2).

For the treatment of estrogen-dependent diseases such as breast cancer of female after menopause, etc., tamoxifen has heretofore been used, which is a drug showing an anti-estrogen action by binding to an estrogen receptor. However, this drug involves a problem in occurrence of resistance, so that, at present, an aromatase inhibitor which is a rate-limiting enzyme participating estrogen synthesis has attracted attention as a treatment agent (Non-Patent Literatures 1 and 2).

The aromatase is a rate-limiting enzyme, which participates at a final stage of sex hormone biosynthesis by aromatizing an A ring of steroid skeleton of a male hormones, i.e., androgens (androstenedione, testosterone) to convert it into a female hormones, i.e., estrogens (estrone, estradiol) (FIG. 1).

Ovaries are the main source of estrogens in female, however, they are synthesized by androgens from adrenal glands via aromatase in extragonadal sites such as muscle and fat in postmenopausal women. Thus, particularly in a hormone therapy of breast cancer after menopause, aromatase has attracted attention as a target enzyme thereof.

With regard to the above-mentioned aromatase inhibitors which used for treating breast cancer after menopause, etc., mostly, they are synthesized compounds. Aromatase inhibitors available in commerce or under clinical testing are roughly classified into Type 1 (steroidal) and Type 2 (non-steroidal) from their structures (FIG. 2).

However, these synthesized compounds involve a possibility to cause Stevens-Johnson syndrome in addition to side effects such as liver disorder live injury or pain at the administered portion, etc. for a clinical use, so that there is a problem that it is necessary to carry out a periodic medical examination during administration (Non-Patent Literature 3).

Accordingly, it has been desired to develop medicine or healthy foods which are expected to have treatment or preventive effects from natural materials with safe and/or without side effects. Moreover, the discovery novel leading compounds for new drug development has also been expected based on such materials.

However, few studies are carried out about aromatase inhibitors from a natural materials such as a crude drugs or a plants, which contain a variety of components (Non-Patent Literature 4).

On the other hand, in recent years, for men, the so-called “male menopausal disorders” which occurs at middle and old age or thereafter is a problem.

The “menopausal disorders” have heretofore been considered to be diseases specific for women, but in recent years, it has been recognized that men also have menopausal disorders. In symptoms of male menopausal disorders, there are symptoms of easily fatigued, depression, decrease in sexuality, etc., and a cause thereof is considered to be decrease in male hormones due to aging (Non-Patent Literature 5).

Clinically, although testosterone replacement therapy has been carried out for the treatment of andropause, the side effects such as liver injury, prostate cancer and loss of hair, etc., are concerned. (Non-Patent Literature 6).

Thus, the present inventors have targeted at aromatase which is an enzyme of convertings the above-mentioned male hormones to a female hormones, and considered that might prevent from decrease of male hormones by inhibiting this enzyme.

It has been reported that in men a conversion rate of from a male hormone to a female hormone is increased with aging (Non-Patent Literature 7), and by administering an aromatase inhibitor to men who suffered from hypogonadism or male hormone deficiency, a testosterone level in blood is recovered or increased (Non-Patent Literature 8, Patent Literature 1).

Moreover, in recent years, at a middle and old age or thereafter which correspond to male menopausal age, in addition to the above-mentioned various symptoms such as easily fatigued, depression, decrease in sexuality, etc., an upper part type fat accumulation pattern, i.e., accumulation of visceral fats is admitted with high frequency, so that it has been attracted attention of a correlation with occurrence of metabolic syndrome. Namely, it has been suggested that the decrease in male hormones (such as testosterone, etc.) with aging should be one of causes in metabolic syndrome (Non-Patent Literature 9).

Also, in men, it has been known that aromatase is distributed in visceral fat in a larger amount as compared with the other portions, and the activity of this enzyme is increased with aging (Non-Patent Literature 10). Moreover, it has been also known that a reverse correlation is shown between the degree of accumulation of visceral fats and testosterone level in blood. From these facts, aromatase in visceral fat is considered to act as an important role in accumulation of fats on internal organs caused by decrease in an amount of testosterone (Non-Patent Literature 11).

Incidentally, replacement of testosterone to aged men decreases the amount of body fat, leptin level in blood and intake amount of meals, and accelerates basal metabolism. Also, in men suffered from hypogonadism, significant increase in an amount of body fat can be admitted with aging, and it has been reported that the amount of body fat is decreased by administration of testosterone (Non-Patent Literature 12).

Thus, in order to seek drugs which shows treatment and/or preventive effects against not only breast cancer of postmenopausal women but also sex hormone-dependent diseases such as male menopausal disorders and metabolic syndrome due to accumulation of visceral fats, etc., the present inventors have carried out search for novel materials having an aromatase-inhibitory activity by aiming at the above-mentioned aromatase.

Non-Patent Literature 1: Chen S, Aromatase and breast cancer, Frontiers in Bioscience, 3; d922-933, 1998.
Non-Patent Literature 2: Simpson E R and Dowsett M., Aromatase and its inhibitors: significance for breast cancer therapy, Recent Prog Horm Res., 57; 317-38, 2002.

### Non-Patent Literature 3: Isomura Yasuo, Okada Minoru, Movement of Research and Development on Aromatase Inhibitor, Pharmacia, vol. 30, 754-758, 1994.

Non-Patent Literature 4: Dietmar G, Gerhard S., Aromatase inhibitors from Urtica dioica Roots, Planta Med., 61; 138-140, 1995. Kim D S, Jeong, H J, et al., Aromatase and sulfatase inhibitors from Lepiota americana, Planta Med., 66; 78-79, 2000. Elizabeth T E, Dudley W, Usha M, et al., Suppression of aromatase (estrogen synthetase) by red wine phytochemicals, Breast Cancer Research and Treatment, 67; 133-146, 2001. Filleur F, Le bail J C, et al., Antiproliferative, anti-aromatase, anti-17β-HSD and antioxidant activities of lignans isolated from Myritica argentea, Planta Med., 67; 700-704, 2001. Minami T, Iwamoto M, Ohtus H, et al., Aromatase inhibitory activities of standishinal and the diterpenoid from the bark of Thuja standishii, Planta Med., 68; 742-745, 2002.
Non-Patent Literature 5: Lamberts S W J, et al., The endocrinology of aging, Science, 278; 419-424, 1997. Itoh Naoki, Tsukamoto Taiji, General concept of andropause, Journal of Clinical and Experimental Medicine (IGAKU NO AYUMI), vol. 205; 380-383, 2003. Iwamoto Teruaki, et al., Reference Ranges of Serum Total and Free Testosterone in Japanese Male Adults, The Japanese Journal of Urology, vol. 95; 751-760, 2004.
Non-Patent Literature 6: Usui Tsuguru, Matsubara Akio, Androgen replacement therapy in PADAM, Journal of Clinical and Experimental Medicine (IGAKU NO AYUMI), vol. 205; 407-410, 2003. Sato Yoshikazu, Tanda Hitosh, Androgen replacement therapy-Clinical indications and problems, Clinic All-Around, vol. 53; 451-457, 2004.
Non-Patent Literature 7: Braunstein G D, Aromatase and gynecomastia, Endocrione-Related Cancer, 6; 315-324, 1999.
Non-Patent Literature 8: Leder B Z, et al., Effects of aromatase inhibitor in elderly men with low or borderline-low serum testosterone levels, J Clin Endocrinol Metab., 89; 1174-1180, 2004. de Boer H, et al., Letrozole normalizes serum testosterone in severely obese men with hypogonadotropic hypogonadism, Diabetes Obes Metab, 7; 211-215, 2005. Raman J D, et al., Aromatase inhibitors for male infertility, The Journal of Urology, 167; 624-629, 2002.
Non-Patent Literature 9: Kawa Gen, Matsuda Tadashi Metabolic syndrome and Male hypogonadism, The Medicinal Frontline, vol. 61; 227-233, 2006. Lunenfeld B., Testosterone deficiency and the metabolic syndrome, The Aging Male, 10; 53-56, 2007. Non-Patent Literature 10: Seidell J, Bjorntorp P, et al., Visceral fat accumulation is positively associated with insulin, glucose and C-peptide levels but negatively with testosterone levels, Metabolism, 39; 897-901, 1990. Kyle U G, Genton L, et al., Age-related differences in fat-free mass, skeletal muscle, body cell mass and fat mass between 18 and 94 years, Eur J Clin Nutr., 55; 663-672, 2001. Bjorntorp P, Adipose tissue distribution and function, International Journal of Obesity, 15; 67-81, 1991. Grooren L, Visceral obesity, the metabolic syndrome, androgens and estrogens, The Aging Male, 9; 75-79, 2006. Grooren L, Toorians A W, Significance of estrogens in male (patho)physiology, Ann Endocrinol., 64; 126-135, 2003.
Non-Patent Literature 11: Cohen P G, The hypogonadal-obesity cycle: role of aromatase in modulating the testosterone-estradiol shunt-a major factor in the genesis of morbid obesity, Med Hypotheses, 52; 49-51, 1999. Cohen P G, Holbrook J M, Other pathways to the manifestations of the Metabolic Syndrome in males, Obesity Research, 12; 1536, 2004.
Non-Patent Literature 12: Rebuffe-Scrive M, Marin P, Bjorntorp P, Effect of testosterone on abdominal adipose tissue in men, Int J. Obes., 15; 791-795, 1991. Allan C A, Strauss B J, et al., Testosterone therapy prevents gain in visceral adipose tissue and loss of skeletal muscle in non-obese aging men, J Clin Endocrinol Metab., 2007 Oct. 16, [Epub ahead of print]. Schroeder E T, Zheng L, et al., Effects of androgen therapy on adipose tissue and metabolism in older men, J Clin Endocrinol Metab., 89; 4863-4872, 2004. Syder P J, et al., Effects of testosterone replacement in hypogonadal men, J Clin Endocrinol Metab., 85; 2670-2677, 2000.

### Patent Literature 1: JP H10-505848 A

### DISCLOSURE OF THE INVENTION

### Problems to be Solved by the Invention

For the treatment of breast cancer of female after menopause, tamoxifen which is a drug showing an anti-estrogen action by binding to estrogen receptor of breast cancer cells has heretofore been used, but in such a case, there is a problem of recurrence of breast cancer due to appearance of resistance. On the other hand, in recent years, an aromatase inhibitor which inhibits estrogen synthesis from male hormones has attracted attention as an effective treatment agent against estrogen-dependent diseases such as the above-mentioned breast cancer, etc. However, in Japan, few aromatase inhibitors which has been admitted to use for clinical purpose, and their use thereof are limited due to various side effects.

On the other hand, male menopausal disorders caused by decrease in testosterone are recently concerned about. Also, a large concern is socially attracted to metabolic syndrome due to accumulation of visceral fats. Moreover, in men at middle and old age or thereafter which correspond to male menopausal stage, it has been suggested that decrease in male hormone such as testosterone, etc., would relate to accumulation of visceral fats. However, there scarcely exists a treatment or preventive effective for such male menopausal disorders or metabolic syndrome due to accumulation of visceral fats.

The object of the present invention is to provide a drug which is effective and safe for treatment and/or prevention of not only breast cancer of female after menopause but also sex hormone-dependent diseases such as male menopausal disorders and metabolic syndrome due to accumulation of visceral fats, etc., by aiming at aromatase which is an enzyme limiting the final stage of biosynthesis of a male hormone and female hormone as a target.

### Means to Solve the Problems

In view of the above-mentioned circumstances, the present inventors have carried out research studies extensively to solve the above-mentioned problems. As a result, they have found that 37 kinds of crude drugs showed inhibitory activity against aromatase, i.e., “Kokeiten” (golden root), prunella spike, sweet hydrangea leaf, milk thistle, a jasmine tea, “Bokusoku” (a bark of Quercus acutissima or a closely related plant thereof), “Tencha” (sweet tea), “Karensou” (Eclipta prostrata), “Youbaihi” (a bark of Myrica rubra Sie b. et Zucc.), French maritime pine, betal palm, asparagus, “Rouro” (a root of Rhaponticum unifloru m DC. or Echinops latifolius Tausch), “Ryoukyou” (a rhizome of Alpinia officinarum Hance), rooibos tea, rhubarb, pu-erh tea, green tea, “Ougon” (a root of Scutellaria baicalensis Georgi), St. John's wort (Hypericum perforatum L.), licorice, “Senrikou” (Senecio scandens Buch.-Ham.), wintergreen, “Kashi” (a matured fruit of Terminalia chebula Retz.), “Yagotou” (a bark of Mallotus japonicus), polygnum root, barrenwort (Epimedium Herb), guarana, “Ouhi” (a bark of Prunus jamasakura Sieb. ex. Koidz, or a closely related plant thereof), Argy's wormwood, sticky rehmannia, Japanese cornel, Asiasarum root, cinnamon, peony root, pine needle, amla fruit, whereby the present invention has accomplished. Moreover, they have also found out aromatase-inhibitory activities on icariin which is a component of barrenwort (Epimedium Herb), and on silybin and silymarin which are components of milk thistle similarly.

That is, the present invention is

1. an aromatase inhibitor which comprises an extract of one or more crude drugs selected from the group consisting of “Kokeiten” (golden root), prunella spike, sweet hydrangea leaf, milk thistle, a jasmine tea, “Bokusoku” (a bark of Quercus acutissima or a closely related plant thereof), “Tencha” (sweet tea), “Karensou” (Eclipta prostrata), “Youbaihi” (a bark of Myrica rubra Sie b. et Zucc.), French maritime pine, betal palm, asparagus, “Rouro” (a root of Rhaponticum unifloru m DC. or Echinops latifolius Tausch), “Ryoukyou” (a rhizome of Alpinia officinarum Hance), rooibos tea, rhubarb, pu-erh tea, green tea, “Ougon” (a root of Scutellaria baicalensis Georgi), St. John's wort (Hypericum perforatum L.), licorice, “Senrikou” (Senecio scandens Buch.-Ham.), wintergreen, “Kashi” (a matured fruit of Terminalia chebula Retz.), “Yagotou” (a bark of Mallotus japonicus), polygnum root, barrenwort (Epimedium Herb), guarana, “Ouhi” (a bark of Prunus jamasakura Sieb. ex. Koidz, or a closely related plant thereof), Argy's wormwood, sticky rehmannia, Japanese cornel, Asiasarum root, cinnamon, peony root, pine needle, amla fruit and an extract thereof.
2. A therapeutic and/or prophylactic agent of sex hormone-dependent diseases which comprises the aromatase inhibitor described in 1.

### EFFECTS OF THE INVENTION

The aromatase inhibitor of the present invention controls decrease in male hormones or increase in female hormones by inhibiting an aromatase which is an enzyme limiting the final stage of biosynthesis of male hormones and female hormones, whereby it can effectively carry out treatment and/or prevention of not only breast cancer of female after menopause, but also of sex hormone-dependent diseases such as male menopausal disorders and metabolic syndrome due to accumulation of visceral fats, etc. Also, the aromatase inhibitor of the present invention can be used safe without any side effects since it comprises a component(s) derived from crude drugs comprising natural materials.

### BEST MODE TO CARRY OUT THE INVENTION

The present inventors have researched to find out a substance which inhibits an aromatase activity among various kinds of natural materials, and as a result, they have found out that an extract of “Kokeiten” (golden root), prunella spike, sweet hydrangea leaf, milk thistle, a jasmine tea, “Bokusoku” (a bark of Quercus acutissima or a closely related plant thereof), “Tencha” (sweet tea), “Karensou” (Eclipta prostrata), “Youbaihi” (a bark of Myrica rubra Sie b. et Zucc.), French maritime pine, betal palm, asparagus, “Rouro” (a root of Rhaponticum unifloru m DC. or Echinops latifolius Tausch), “Ryoukyou” (a rhizome of Alpinia officinarum Hance), rooibos tea, rhubarb, pu-erh tea, green tea, “Ougon” (a root of Scutellaria baicalensis Georgi), St. John's wort (Hypericum perforatum L.), licorice, “Senrikou” (Senecio scandens Buch.-Ham.), wintergreen, “Kashi” (a matured fruit of Terminalia chebula Retz.), “Yagotou” (a bark of Mallotus japonicus), polygnum root, barrenwort (Epimedium Herb), guarana, “Ouhi” (a bark of Prunus jamasakura Sieb. ex. Koidz, or a closely related plant thereof), Argy's wormwood, sticky rehmannia, Japanese cornel, Asiasarum root, cinnamon, peony root, pine needle, or amla fruit inhibits an aromatase activity. Details of these crude drugs are as follows.

(1) “Kokeiten” (golden root) is a material in which a whole petal of Kokeiten (golden root) (Rhodiola sacra Fu) belonging to an orpine family (Crassulaceae) or an underground portion of the other plants belonging to the same genus is dried.
(2) Prunella spike (“Kagoso”) is a material in which a fruit-spike of self-heal (Prunella vulgaris L. var. lilacina Nak.) belonging to a perilla family (Labiaceae) is dried.
(3) Sweet hydrangea leaf (hortensia) is a material in which young leaves of hortensia (Hydrangea macrophylla var. thunbergii) which is a variant of a low deciduous tree Hydrangea macrophylla Ser. F. normalis belonging to a saxifrage family (Saxifragaceae) are steamed, rubbed and dried.
(4) Milk thistle is a material in which a dry fruit of milk thistle (Silybum marianum L., another name: Carduus marianus L.) belonging to a chrysanthemum family (Asteraceae) is dried.
(5) A jasmine tea is a material in which green tea and a flower petal of jasmine (Jasminum sambac (L.) Ait.) are mixed and dried.
(6) “Bokusoku” (a bark of Querus acutissima or a closely related plant thereof) is a material in which a bark of a kind of oak (Querus acutissima) belonging to a beech family (Fagaceae) or other analogue plants is dried.
(7) “Tencha” (sweet tea) is a material in which young leaves of “Rouren Shukyu” (Hydrangea strigosa Rehd.) or Yakushima hydrangea (Hydrangea umbellate Rehd.) belonging to a saxifrage family (Saxifragaceae) are dried.
(8) “Karensou” (Eclipta prostrata) is a material in which whole plant of “Takasaburo” (Eclipta prostrata) belonging to a chrysanthemum family (Compositae) is dried.
(9) “Youbaihi” (a bark of Myrica rubra Sie b. et Zucc.) is a material in which a bark of bayberry (Myrica rubra Sieb. et Zucc.) belonging to a bayberry family (Myricaceae) is dried.
(10) French maritime pine is a material in which a bark of Pinus pinaster or P. maritima belonging to a pine family (Pinaceae) is dried.
(11) Betal palm (“Binro”) is a material in which seeds of areca palm (Areca catechu L.) which is a plant belonging to a palm family (Arecaceae) are dried.
(12) Asparagus is a material in which a rhizome or root of common asparagus (Asparagus officinalis L.) belonging to a lily family (Liliaceae) is dried.
(13) “Rouro” (a root of Rhaponticum unifloru m DC. or Echinops latifolius Tausch) is a material in which a root of “Kishu Rouro” (a root of Rhaponticum unifloru m DC. or Echinops latifolius Tausch) (Rhaponticum uniflorum DC.) “Ohrurihigotai” (Echinops latifolius Tausch) belonging to a chrysanthemum family (Compositae) is dried.
(14) “Ryoukyou” (a rhizome of Alpinia officinarum Hance) is a material in which a rhizome of lesser galangal root (Alpinia officinarum Hance) belonging to a ginger family (Zingiberaceae) is dried.
(15) Rooibos tea is a material in which leaves like conifer of Aspalathus linearis belonging to a bean family (Leguminosae) is dried.
(16) Rhubarb (“Daiou”) is a material in which a rhizome of Chinese rhubarb (Rheum palmatum L.), Tangute (Toukotoku) rhubarb (R. tanguticum Maxim. ex Rgl.), Chinese (medical) rhubarb (R. officinale Baillon) or R. coreanum Nakai belonging to a smartweed family (Polygonaceae) or a hybrid between these species is dried.
(17) Pu-erh tea is a material in which green tea oxidation-fermentation of which is stopped by heating is fermented with Aspergillus and dried.
(18) Green tea is a material in which leaves of a tree of tea (Camellia sinensis Kunt) belonging to a camellia family (Theaceae) are dried.
(19) “Ougon” (a root of Scutellaria baicalensis Georgi) is a material in which a root of Chinese skullcap (Scutellaria baicalensis Georgi) belonging to perilla family (Labiatae).
(20) St. John's wort (Hypericum perforatum L.) is a material in which herb (an upperground portion) of St. John's wort (Hypericum perforatum L.) belonging to a Jinsitao family (Hypericaceae) is dried.
(21) Licorice (glycyrrhiza) is a material in which a root and stolon of Chinese licorice (Glycyrrhiza uralensis Fisch.) or European licorice (G. glabra L.) belonging to a bean family (Leguminosae) are dried.
(22) “Senrikou” (Senecio scandens Buch.-Ham.) is a material in which whole plant of charlatan ivy (Senecio scandens Buch.-Ham.) belonging to a chrysanthemum family (Compositae) is dried.
(23) Wintergreen is a material in which leaves of Gaultheria procumbens belonging to an azalea family (Ericaceae) are dried.
(24) “Kashi” (a matured fruit of Terminalia chebula Retz.) is a material in which a matured fruit of Terminalia chebula Retz. belonging to a Rangoon creeper family (Combretaceae) is dried.
(25)“Yagotou” (a bark of Mallotus japonicus) is a material in which a bark of Japanese Mellotus (Mallotus japonicus) belonging to a sun spurge (Euphorbia helioscopia) family (Euphorbiaceae) is dried.
(26) Polygnum root (“Kashuu”) is a material in which a tuber of Chinese knotweed (Polygonum multiflorum Thunberg) belonging to a smartweed family (Polygonaceae) is dried.
(27) Barrenwort (Epimedium Herb) is a material in which stem and leaves of Epimedium pubescens Maximowicz, E. brevicornum Maximowicz, E. wushanense T. S. Ying, sagittatum (E. sagittatum Maximowicz), koreanum (E. koreanum Nakai), horny goat weed (E. gradiflorum Morr.) or sempervirens (E. sempervirens Nakai) belonging to a barbery family (Berberidaceae) are dried.
(28) Guarana is a material in which seeds of Paullina cupana H. B. K belonging to a soapberry tree or Soapnut-tree family (Sapindaceae) are dried.
(29) “Ouhi” (a bark of Prunus jamasakura Sieb. ex. Koidz, or a closely related plant thereof) (bark of cherry tree) is a material in which a bark of a plant of wild cherry tree (Prunus jamasakura Sieb. ex Koidz.) belonging to a rose family (Rosaceae) or an analogous plant belonging to the same genus is dried.
(30) Argy's wormwood (“Gaiyou”) is a material in which whole plant or leaves of mugwort (Artemisia princeps Pamp.) or wild mugwort (A. montana Pamp.) belonging to a chrysanthemum family (Compositae) are dried.
(31) Sticky rehmannia (“Jiou”) is a material in which a root of glutinous rehmannia (Rehmannia glutinosa Liboschitz var. purpurea Makino) or R. glutinosa Liboschitz belonging to a snapdragon family (Scrophulariaceae) is dried.
(32) Japanese cornel (dogwood tree) is a material in which the flesh of fruit of an accessory fruit of dogwood tree (Cornus officinalis Siebold et Zuccarini) belonging to a dogwood family (Cornaceae) is dried.
(33) Asiasarum root (wild ginger, “Saishin”) is a material in which a root and rhizome of Siebold's wild ginger (Asiasarum sieboldii Miq.) or Chinese wild ginger (A. heterotropoides F. Schm. var. mandshuricum F. Maekawa) belonging to a Dutchman's pipe family (Aristolochiaceae) is dried.
(34) Cinnamon (cassia bark) is a material in which a bark of cassia-bark-tree (Cinnamomum cassia Blume) belonging to a camphor tree family (Lauraceae) is dried.
(35) Peony root (“Shakuyaku”) is a material in which a root of herbaceous peony (Paeonia lactiflora Pall.) belonging to a peony family (Paeoniaceae) is dried.
(36) Pine needle (“Matsuba”) is a material in which leaves of Japanese red pine (Pinus densiflora Sieb. et Zucc.) or Japanese black pine (P. thunbergii Parl.) belonging to a pine family (Pinaceae) are dried.
(37) Amla fruit is a material in which a fruit of Emblica officinalis Gaertn belonging to a sun spurge (Euphorbia helioscopia) family (Euphorbiaceae) is dried.

Also, of these crude drugs, chemical structures of icariin which is a conventionally known component contained in barrenwort (Epimedium Herb), and silybin and silymarin which are conventionally known components contained in milk thistle are as follows: Incidentally, silymarin is a mixture of flavanone derivatives such as silybin, silychristin, silydiani, etc.

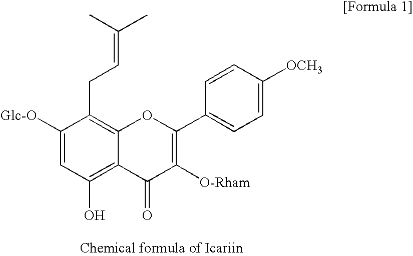

(wherein Rham and Glc each represent lamnose and glucose residue)

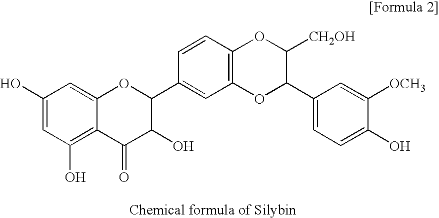

As the portions to be used of the respective plants which become a material of crude drugs to be used in the present invention, those mentioned above are preferred, and in addition to the above, one or more portions selected from flower, spike, fruit peel, fruit, stem, leaf, twig, branches and leaves, trunk, bark, rhizome, root bark, root, seed or whole plant, etc., can be used. As the extract, in addition to the material obtained by directly extracting these various kinds of crude drugs using a solvent, a compressed solution obtained by subjecting to compression treatment and/or a material obtained by adding a solvent to a residue and extracting is/are also included in the scope of the definition of the extracts according to the present invention.

The extract of the crude drugs according to the present invention may be those prepared by the conventionally known methods, for example, they can be prepared by using an extraction solvent such as water, alcohols including methanol, ethanol, etc., or a mixed solvent of these solvents, and carrying out normal temperature extraction or extraction under heating, and, if necessary, extraction may be carried out under reduced pressure or under pressure. The obtained extract can be used as such, but in general, a material obtained by concentration or concentrated to dryness by lyophilization (freeze-drying) is used.

### EXAMPLES

In the following, the present invention is explained by referring to extraction examples, but the present invention is not limited to these examples.

### Example 1

### Extraction Method of Crude Drugs (1)

To each 100 g of dried product of prunella spike (fruit spike), sweet hydrangea leaf (leaf), a jasmine tea (leaf, flower), “Bokusoku” (a bark of Quercus acutissima or a closely related plant thereof) (bark), “Tencha” (sweet tea) (leaf), “Karensou” (Eclipta prostrata) (whole plant), “Youbaihi” (a bark of Myrica rubra Sie b. et Zucc.) (bark), French maritime pine (bark), betal palm (seed), “Rouro” (a root of Rhaponticum unifloru m DC. or Echinops latifolius Tausch) (root), “Ryoukyou” (a rhizome of Alpinia officinarum Hance) (rhizome), rooibos tea (leaf), rhubarb (root), pu-erh tea (leaf), green tea (leaf), “Ougon” (a root of Scutellaria baicalensis Georgi) (rhizome), licorice (root), “Senrikou” (Senecio scandens Buch.-Ham.) (whole plant), wintergreen (leaf), “Kashi” (a matured fruit of Terminalia chebula Retz.) (fruit), “Yagotou” (a bark of Mallotus japonicus) (rind), polygnum root (tuber), barrenwort (Epimedium Herb) (leaf), “Ouhi” (a bark of Prunus jamasakura Sieb. ex. Koidz, or a closely related plant thereof) (rind), Argy's wormwood (whole plant), sticky rehmannia (root), Japanese cornel (flesh of fruit), Asiasarum root (root), cinnamon (rind), peony root (root), pine needle (leaf) was added each 300 L of purified water, extraction was carried out at 80° C. for 1 hour twice under reflux, and a filtered extract was lyophilized (freeze-dried) according to the conventional method. As a result, each 18.5 g, 31.0 g, 27.3 g, 31.2 g, 13.8 g, 17.6 g, 20.4 g, 23.6 g, 17.8 g, 21.5 g, 22.0 g, 19.8 g, 35.5 g, 14.9 g, 12.7 g, 32.1 g, 33.7 g, 14.3 g, 15.1 g, 21.4 g, 24.3 g, 35.3 g, 15.2 g, 21.1 g, 12.3 g, 33.6 g, 30.8 g, 29.3 g, 25.1 g, 22.1 g, 15.0 g was obtained as a dried solid content.

### Example 2

### Extraction Method of Crude Drugs (2)

To 100 g of a dried material of Asparagus (rhizome, root) was added 300 L of 30% ethanol/purified water, to 100 g of a dried material of guarana (seed) was added 300 L of 35% ethanol/purified water, to 100 g of a dried material of “Kokeiten” (golden root) (underground portion) was added 300 L of 50% ethanol/purified water, to each 100 g of St. John's wort (Hypericum perforatum L.) (herb (upper ground portion)) dried material and amla fruit (fruit) dried material was each added 300 L of 60% ethanol/purified water, and to 100 g of a dried material of milk thistle (fruit peel) was added 300 L of 80% ethanol/purified water, extraction was each carried out at 80° C. for 1 hour twice under reflux, and a filtered extract was lyophilized (freeze-dried) according to the conventional method. As a result, each 16.4 g, 17.9 g, 30.3 g, 27.5 g, 26.3 g and 3.5 g was obtained as a dried solid content.

### Example 3

### Compound to be Tested

With regard to icariin which is a conventionally known component contained in barrenwort (Epimedium Herb), and silybin and silymarin which are conventionally known components contained in milk thistle, commercially available standard products were used.

That is, icariin (LKT, Laboratories, Inc, USA, Lot No. 2591307), silybin (Extrasynthese, France, Lot No. 02112642) and silymarin (LKT, Laboratories, Inc, USA, Lot No. 2397805) were used.

### Example 4

### Measurement of Aromatase-Inhibitory Activity

Measurement of an aromatase-inhibitory activity was carried out based on the method published by an already known literature (Sresser D M, Tuner S D, et al., A High-throughput screen to identify inhibitors of aromatase (CYP19), Analytical Biochemistry, 284; 427-430, 2000.), using reagents available from BD Biosciences Inc. (U.S.A.). As Comparative example (positive control), chrysin (Extrasynthese, France, Lot No. 06042506) was used (FIG. 2).

That is, by using a 96-well microplate, after 144 μL of a previously prepared NADPH-producing system solution (NADPH-Cofactor Mix) and 6 μL of an extract solution of test samples were mixed, and incubated at 37° C. for 10 minutes. Then, 100 μL of a solution (Enzyme Substrate Mix) containing an enzyme and a substrate was mixed with the above mixture, and reacted at 37° C. for 30 minutes. Thereafter, 75 μL of a reaction-terminating solution was added to the above, and an amount of a formed HFC (7-hydroxy-4-trifluoromethyl coumarin) which is a metabolite of the substrate was obtained by measuring a fluorescent intensity at an excitation wavelength of 409 nm and a fluorescent wavelength of 538 nm using a plate reader (SPECTRAFluor, TECAN). Incidentally, 75 μL of stop solution was added to a blank after incubation for 10 minutes, in place of adding the solution of enzyme and substrate. Aromatase inhibitory rate was calculated by the formula 1.

Aromatase inhibitory rate (%)={1−(A−B)/A}×100 [Numerical formula 1]

A=(Absorbance after enzymatic reaction without test sample−Absorbance of blank thereof)
B=(Absorbance after enzymatic reaction with each test sample−Absorbance of each blank thereof)

Also, to avoid a non-specific inhibitory action (for example, protein coagulation action due to tannin), a control protein which incidents to the reagent was added to the NADPH-producing system solution.

With regard to solution of test sample, the concentration was stepwisely diluted and an inhibitory rate at each concentration was obtained, and from the results, the concentration of 50% inhibition (IC₅₀ value) was obtained according to the interpolation method.

### [Test Results]

With regard to about 400 kinds of extracts, aromatase-inhibitory activities were measured. As a result, an inhibitory activity with concentration-dependent and of 50% or higher at a maximum concentration of 100 μg/mL was admitted in the following 37 kinds of crude drug extracts. In Tables 1 to 4, IC₅₀ values of these crude drug extracts were shown in the order of potent inhibitory activities.

That is, those in which inhibitory activities are admitted are extracts of, in total of 37 kinds:

“Kokeiten” (golden root), prunella spike, sweet hydrangea leaf, milk thistle, a jasmine tea, “Bokusoku” (a bark of Quercus acutissima or a closely related plant thereof), “Tencha” (sweet tea), “Karensou” (Eclipta prostrata), “Youbaihi” (a bark of Myrica rubra Sie b. et Zucc.), French maritime pine, betal palm, asparagus, “Rouro” (a root of Rhaponticum unifloru m DC. or Echinops latifolius Tausch), “Ryoukyou” (a rhizome of Alpinia officinarum Hance), rooibos tea, rhubarb, pu-erh tea, green tea, “Ougon” (a root of Scutellaria baicalensis Georgi), St. John's wort (Hypericum perforatum L.), licorice, “Senrikou” (Senecio scandens Buch.-Ham.), wintergreen, “Kashi” (a matured fruit of Terminalia chebula Retz.), “Yagotou” (a bark of Mallotus japonicus), polygnum root, barrenwort (Epimedium Herb), guarana, “Ouhi” (a bark of Prunus jamasakura Sieb. ex. Koidz, or a closely related plant thereof), Argy's wormwood, sticky rehmannia, Japanese cornel, Asiasarum root, cinnamon, peony root, pine needle and amla fruit, and the respective IC₅₀ values were 6.1 μg/mL, 7.4 μg/mL, 7.4 μg/mL, 7.7 μg/mL, 7.8 μg/mL, 8.9 μg/mL, 9.0 μg/mL, 10.1 μg/mL, 10.3 μg/mL, 10.7 μg/mL, 11.3 μg/mL, 13.2 μg/mL, 13.4 μg/mL, 15.2 μg/mL, 16.0 μg/mL, 17.2 μg/mL, 17.8 μg/mL, 17.8 μg/mL, 20.1 μg/mL, 21.2 μg/mL, 22.7 μg/mL, 23.5 μg/mL, 26.9 μg/mL, 27.7 μg/mL, 30.1 μg/mL, 31.9 μg/mL, 35.0 μg/mL, 37.6 μg/mL, 37.8 μg/mL, 40.4 μg/mL, 44.7 μg/mL, 52.0 μg/mL, 58.0 μg/mL, 59.3 μg/mL, 72.5 μg/mL, 78.9 μg/mL and 98.4 μg/mL (see Tables 1 to 4).

These 37 kinds of crude drugs have been used in China and other countries than China many years ago, but it has never been known that these drugs have aromatase-inhibitory activities as of today, and this is novel findings firstly obtained by the present invention.

Incidentally, with regard to Pueraria root (Kakkon), Immature Orange (Kijitsu), Jujube fruit (Taisou), heavenly bamboo seed (Nandia domestica), bellflower (Platycodon grandiflorus), Magnolia bark (Kouboku), Bupleurum root (Saiko), Citrus Unshiu peel (Chinpi), Cuscuta chinensis (Toshishi), Ophiopogon tuber (Bakumondou), Sinomenium stem (Boui), Schizonepeta tenuifolia (Keigai), Poria Sclerotium (Bukuryou), processed aconite root (Bushi), cordyceps (Cordyceps sinensis) (Touchuu Kasou), Alisma Rhizome (Takusha), Dioscore Rhizome (San-yaku), Nux Vomica (Strychni Semen), Cyperus Rhizome (Koubushi), Magnolia Flower (Shin-i), Eleutherococcus Senticosus Rhizome (Shigoka), Red Ginseng (Koujin), Panax Japonicus Rhizome (Chikusetu Ninjin), Eucommia bark (Tochuu), Atractylodes Rhizome (Byakujutsu), Atractylodes Lancea Rhizome (Soujutsu), Japanese valerian (Kanokosou), Agkistrodon Japonicae (Hanbi), ginger, Senega (Senegae Radix), hop, red grape leaf, Agni, European pumpkin seed, willow bark, chamomile, nettle, pepper mint, olive leaf, millet seed, burdock fruit, Siberian ginseng, European dandelion (Taraxacum officinale), Artichoke or Globe artichoke (Cynara scolymus), garlic, melissa leaf, leek seed, pomegranate seed, European hawthorn (Crataegus oxyacantha), European willow, celery seed, thyme, lavender, hibiscus, rose hip, rosemary, Cucurbita moscbata Duch (Nankashi), oat, eyebright (Euphrasia officinalis), bitter melon (Momordica charantia var. pavel), Daphne genkwa (Thymelaeaceae), corn whiskers (Gyokubeishu), Cynanchum paniculatum Kitag. (Jochoukyou), Japanese Angelica Root's leaf (Toukiba), Bombyx Batryticatus (Byakkyousan), Eleutherococcus senticosus (Ezoukogi), root of Pulsatilla cernua (Hakutouou), molokheiya, Sanguisorbae Radix (Chiyu), duckweed, (Tibetan chaenomeles fruit), mulberry root, unripe tangerine peel (Citri Exocarpium Immaturum), Japanese mahonia leaf (Mahoniae Folium), baiwei cynanchum root (Cynanchi Baiwei Radix), bamboo shavings (Bambusae Caulis in Taeniam), Trichosanthes root (Trichosanthis Radix), Pogonatherum (Pogonatherum crinitum), Yuzu (Citrus junos), wild chrysanthemum flower (Chrysanthemum indicum L.), selaginella (Selafinellae Herba), ash bark (Fraxini Cortex), Japanese Gentian (Ryuutan), adenophora root (Adenophorae Radix), Cannabidis semen (Mashinin), Saposhnikovia root (Boufuu), Japanese spikenard rhizome (Aralia cordata), sophora fruit (Sophorae Fructus), Japanese dandelion, Amomum seed (Shukusha), Phellodendron bark (Oubaku), Corydalis Tuber (Engosaku), Achyranthes root (Goshitsu), Bolbostemma paniculatum (Bolbostemmatis Tuber), Houttuyniae Herba (Juuyaku), belamcandae root (Belamcandae Rhizoma), Cimicifuga Rhizome (Shouma), Mulberry bark (Souhakuhi), madder (Rubia akane), toosendan fruit (Toosendan Fructus), bushy sphora root (Sophorae Subprostratae Radix), silk tree bark, Angelica Dahurica root (Byakushi), Cayenne pepper (Capsicum annuum L.), areca husk (Arecae Pericarpium), Astragalus root (Ougi), Coptis Rhizome (Ouren), Coix seed (Yokuinin), Gardenia Fruit (Sanshishi), Bupleurum root (Saiko), chrysanthemum flower (Kikka), Asian ginseng, etc., aromatase-inhibitory activities thereof were less than 50% at 100 μg/mL, or even when their inhibitory activities were 50% or more at 100 μg/mL, no concentration dependency on the inhibitory activity was admitted.

TABLE 1
Inhibitory activities of 37 kinds of extracts against aromatase - 1

| Substance to be | Concentration | Inhibitory rate | IC₅₀ value |
|---|---|---|---|
| tested | (μg/ml) | (%) | (μg/ml) |
| Chrysin | 1.22 | 71.8 ± 13.3 | 0.28 |
| (Comparative | 0.41 | 61.3 ± 11.0 | |
| example) | 0.14 | 40.5 ± 11.9 | |
| | 0.05 | 16.6 ± 10.0 | |
| Kokeiten: 50% | 100.0 | 100.0 ± 6.9 | 6.1 |
| ethanol extract | 33.3 | 82.1 ± 5.5 | |
| | 11.1 | 75.9 ± 11.7 | |
| | 3.7 | 31.4 ± 4.6 | |
| Prunella spike: | 100.0 | 100.0 ± 0.0 | 7.4 |
| water extract | 33.3 | 100.0 ± 0.0 | |
| | 11.1 | 42.7 ± 13.4 | |
| | 3.7 | 21.4 ± 11.0 | |
| Sweet hydrangea | 100.0 | 84.8 ± 15.2 | 7.4 |
| leaf: water extract | 33.3 | 95.8 ± 11.9 | |
| | 11.1 | 66.0 ± 11.9 | |
| | 3.7 | 21.0 ± 11.7 | |
| Milk thistle: 80% | 100.0 | 87.0 ± 3.0 | 7.7 |
| ethanol extract | 33.3 | 64.9 ± 3.7 | |
| | 11.1 | 57.2 ± 10.1 | |
| | 3.7 | 40.1 ± 9.8 | |
| Jasmine tea: water | 100.0 | 94.4 ± 10.8 | 7.8 |
| extract | 33.3 | 91.2 ± 15.0 | |
| | 11.1 | 54.0 ± 9.1 | |
| | 3.7 | 29.3 ± 8.2 | |
| Bokusoku: water | 100.0 | 96.0 ± 5.0 | 8.9 |
| extract | 33.3 | 96.9 ± 5.4 | |
| | 11.1 | 71.2 ± 4.9 | |
| | 3.7 | 10.4 ± 6.8 | |
| Tencha: water | 100.0 | 100.0 ± 0.0 | 9.0 |
| extract | 33.3 | 87.4 ± 14.0 | |
| | 11.1 | 48.7 ± 14.8 | |
| | 3.7 | 23.3 ± 14.7 | |

Note:
Inhibitory rate is an average value obtained by three times of experiments.

TABLE 2
Inhibitory activities of 37 kinds of extracts against aromatase - 2

| Substance to be | Concentration | Inhibitory rate | IC₅₀ value |
|---|---|---|---|
| tested | (μg/ml) | (%) | (μg/ml) |
| Karensou: water | 100.0 | 99.0 ± 1.4 | 10.1 |
| extract | 33.3 | 94.7 ± 9.2 | |
| | 11.1 | 47.7 ± 14.8 | |
| Youbaihi: water | 100.0 | 75.9 ± 5.0 | 10.3 |
| extract | 33.3 | 88.8 ± 3.3 | |
| | 11.1 | 56.8 ± 4.8 | |
| | 3.7 | 20.6 ± 4.5 | |
| French maritime | 100.0 | 88.3 ± 9.2 | 10.7 |
| pine: water extract | 33.3 | 98.0 ± 3.5 | |
| | 11.1 | 62.7 ± 10.0 | |
| | 3.7 | 8.5 ± 13.2 | |
| Betal palm: water | 100.0 | 83.6 ± 14.1 | 11.3 |
| extract | 33.3 | 91.4 ± 10.2 | |
| | 11.1 | 35.7 ± 7.1 | |
| Asparagus: 30% | 100.0 | 80.1 ± 16.5 | 13.2 |
| ethanol extract | 33.3 | 79.9 ± 8.3 | |
| | 11.1 | 39.2 ± 2.7 | |
| Rouro: water | 100.0 | 81.8 ± 12.1 | 13.4 |
| extract | 33.3 | 83.1 ± 5.9 | |
| | 11.1 | 37.1 ± 8.6 | |
| Ryoukyou: water | 100.0 | 83.1 ± 5.6 | 15.2 |
| extract | 33.3 | 81.0 ± 5.9 | |
| | 11.1 | 34.7 ± 5.8 | |
| Rooibos tea: water | 100.0 | 85.1 ± 20.4 | 16.0 |
| extract | 33.3 | 93.8 ± 5.9 | |
| | 11.1 | 33.5 ± 7.6 | |
| Rhubarb: water | 100.0 | 92.6 ± 2.2 | 17.2 |
| extract | 33.3 | 56.8 ± 3.2 | |
| | 11.1 | 43.4 ± 25.4 | |
| Pu-erh tea: water | 100.0 | 78.0 ± 15.6 | 17.8 |
| extract | 33.3 | 80.9 ± 16.5 | |
| | 11.1 | 37.3 ± 6.8 | |

Note:
Inhibitory rate is an average value obtained by three times of experiments.

TABLE 3
Inhibitory activities of 37 kinds of extracts against aromatase - 3

| Substance to be | Concentration | Inhibitory rate | IC₅₀ value |
|---|---|---|---|
| tested | (μg/ml) | (%) | (μg/ml) |
| Green tea: water | 100.0 | 70.9 ± 9.0 | 17.8 |
| extract | 33.3 | 65.4 ± 7.8 | |
| | 11.1 | 41.6 ± 1.8 | |
| Ougon: water | 100.0 | 86.6 ± 10.9 | 20.1 |
| extract | 33.3 | 55.1 ± 3.2 | |
| | 11.1 | 39.0 ± 7.8 | |
| St. John's wort: | 100.0 | 100.0 ± 0.0 | 21.2 |
| 60% ethanol extract | 33.3 | 72.1 ± 1.1 | |
| | 11.1 | 32.8 ± 8.4 | |
| Licorice: water | 100.0 | 89.1 ± 5.3 | 22.7 |
| extract | 33.3 | 65.4 ± 2.6 | |
| | 11.1 | 25.9 ± 7.2 | |
| Senrikou: water | 100.0 | 61.4 ± 6.6 | 23.5 |
| extract | 33.3 | 78.0 ± 5.4 | |
| | 11.1 | 26.6 ± 4.3 | |
| Wintergreen: water | 100.0 | 78.8 ± 7.4 | 26.9 |
| extract | 33.3 | 72.8 ± 11.8 | |
| | 11.1 | 18.2 ± 8.2 | |
| Kashi: water extract | 100.0 | 70.7 ± 9.3 | 27.7 |
| | 33.3 | 57.1 ± 14.8 | |
| | 11.1 | 32.0 ± 21.3 | |
| Yagotou: water | 100.0 | 73.3 ± 6.4 | 30.1 |
| extract | 33.3 | 41.9 ± 13.1 | |
| | 11.1 | 33.5 ± 3.8 | |
| Polygnum root: | 100.0 | 91.2 ± 0.2 | 31.9 |
| water extract | 33.3 | 33.4 ± 2.1 | |
| | 11.1 | 21.4 ± 9.0 | |
| Barrenwort: water | 100.0 | 86.0 ± 1.6 | 35.0 |
| extract | 33.3 | 38.1 ± 6.7 | |
| | 11.1 | 18.2 ± 12.9 | |

Note:
Inhibitory rate is an average value obtained by three times of experiments.

TABLE 4
Inhibitory activities of 37 kinds of extracts against aromatase - 4

| Substance to be | Concentration | Inhibitory rate | IC₅₀ value |
|---|---|---|---|
| tested | (μg/ml) | (%) | (μg/ml) |
| Guarana: 35% | 100.0 | 73.7 ± 28.8 | 37.6 |
| ethanol extract | 33.3 | 55.5 ± 21.3 | |
| | 11.1 | 14.6 ± 8.7 | |
| Ouhi: water extract | 100.0 | 74.5 ± 10.5 | 37.8 |
| | 33.3 | 40.5 ± 13.5 | |
| | 11.1 | 26.0 ± 2.0 | |
| Argy's wormwood: | 100.0 | 94.2 ± 13.7 | 40.4 |
| water extract | 33.3 | 16.1 ± 1.7 | |
| | 11.1 | 8.9 ± 7.9 | |
| Sticky rehmannia: | 100.0 | 100.0 ± 1.2 | 44.7 |
| water extract | 33.3 | 38.6 ± 4.3 | |
| | 11.1 | 20.6 ± 18.5 | |
| Japanese cornel: | 100.0 | 55.3 ± 13.1 | 52.0 |
| water extract | 33.3 | 48.5 ± 11.7 | |
| | 11.1 | 32.6 ± 26.1 | |
| Asiasarum root: | 100.0 | 100.0 ± 43.3 | 58.0 |
| water extract | 33.3 | 11.1 ± 11.1 | |
| | 11.1 | 5.6 ± 7.3 | |
| Cinnamon: water | 100.0 | 64.0 ± 5.1 | 59.3 |
| extract | 33.3 | 32.0 ± 7.6 | |
| | 11.1 | 20.8 ± 12.1 | |
| Peony root: water | 100.0 | 65.2 ± 4.7 | 72.5 |
| extract | 33.3 | 18.9 ± 2.0 | |
| | 11.1 | 10.6 ± 14.4 | |
| Pine needle: water | 100.0 | 53.2 ± 7.7 | 78.9 |
| extract | 33.3 | 31.5 ± 13.1 | |
| | 11.1 | 4.2 ± 9.3 | |
| Amla fruit: 60% | 100.0 | 50.0 ± 11.8 | 98.4 |
| ethanol extract | 33.3 | 43.5 ± 3.1 | |
| | 11.1 | 36.6 ± 6.9 | |

Note:
Inhibitory rate is an average value obtained by three times of experiments.

Also, when aromatase-inhibitory activities of icariin which is a conventionally known component contained in barrenwort (Epimedium Herb), and silybin and silymarin which are conventionally known components contained in milk thistle are investigated, then, each showed concentration-dependent inhibitory activity, and IC50 value was each 0.754 μM, 4.86 μM and 3.79 μM (Table 5). Incidentally, of these compounds, icariin showed more potent aromatase-inhibitory activity than that of chrysin which is positive control.

Whereas these compounds are each conventionally known component, but it has never been known that they have aromatase-inhibitory activities as of today, and this is novel findings for the first time obtained by the present invention.

TABLE 5
Inhibitory activities of 3 kinds of components against aromatase

| Substance to be | Concentration | Inhibitory rate | IC₅₀ value |
|---|---|---|---|
| tested | (μM) | (%) | (μg/ml) |
| Chrysin | 4.80 | 71.8 ± 13.3 | 1.11 |
| (Molecular weight: | 1.60 | 61.3 ± 11.0 | |
| 254.2) | 0.53 | 40.5 ± 11.9 | |
| | 0.18 | 16.6 ± 10.0 | |
| Icariin | 4.80 | 67.7 ± 21.3 | 0.754 |
| (Molecular weight: | 1.60 | 63.5 ± 12.6 | |
| 676.7) | 0.53 | 48.1 ± 6.9 | |
| | 0.18 | 30.4 ± 1.5 | |
| Silybin | 4.80 | 59.4 ± 7.1 | 4.86 |
| (Molecular weight: | 1.60 | 20.6 ± 1.2 | |
| 482.4) | 0.53 | 12.4 ± 4.6 | |
| | 0.18 | 8.7 ± 9.3 | |
| Silymarin* | 4.80 | 55.2 ± 3.0 | 3.79 |
| (Molecular weight: | 1.60 | 35.1 ± 4.2 | |
| 486.4) | 0.53 | 16.8 ± 8.9 | |
| | 0.18 | 9.4 ± 8.9 | |

*1: Inhibitory rate is an average value obtained by three times of experiments.
*2: The molecular weight of Silymarin was an average value of the molecular weights of silybin, silychristin and silydianin.

### CONCLUSION

With regard to 37 kinds of crude drug extracts in which an aromatase-inhibitory activity was firstly found out by the present invention, medicines or healthy foods containing these materials are considered to contribute to treatment and/or prevention of not only breast cancer of female after menopause, but also male menopausal disorders and sex hormone-dependent diseases such as metabolic syndrome due to accumulation of visceral fats, etc.

Also, with regard to icariin which is a component contained in barrenwort (Epimedium Herb), and silybin and silymarin which are components contained in milk thistle among these crude drugs, it can be expected to provide a leading compound to develop a novel aromatase inhibitor by chemically modifying these compounds.

### BRIEF DESCRIPTION OF THE DRAWINGS

FIG. 1 is a drawing showing a relation between aromatase with androgen (androgens) and female hormone (estrogens).

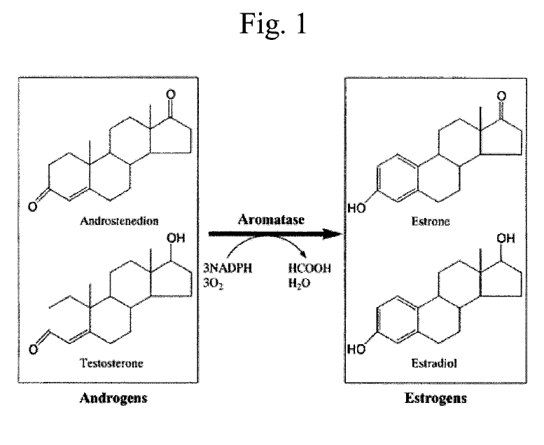

FIG. 2 is a drawing showing a structural formula of an aromatase inhibitor to be used for the treatment of breast cancer of female after menopause.

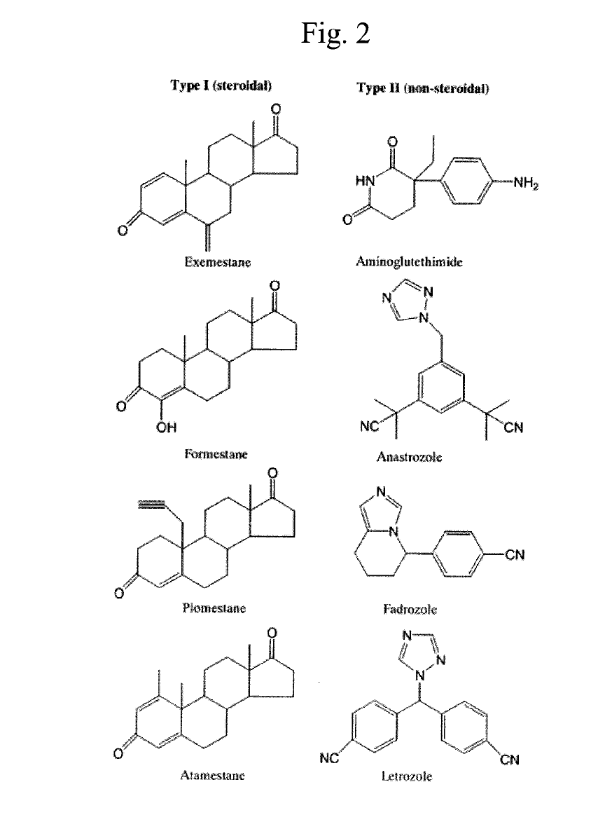

FIG. 3 is a drawing showing a structural formula of chrysin which is Comparative example (positive control) in the present invention.

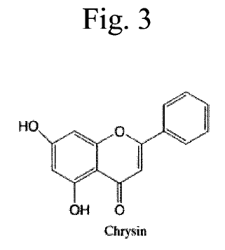

1. An aromatase inhibitor which comprises an extract of one or more crude drugs selected from the group consisting of “Kokeiten” (golden root), prunella spike, sweet hydrangea leaf, milk thistle, a jasmine tea, “Bokusoku” (a bark of Quercus acutissima or a closely related plant thereof), “Tencha” (sweet tea), “Karensou” (Eclipta prostrata), “Youbaihi” (a bark of Myrica rubra Sie b. et Zucc.), French maritime pine, betal palm, asparagus, “Rouro” (a root of Rhaponticum unifloru m DC. or Echinops latifolius Tausch), “Ryoukyou” (a rhizome of Alpinia officinarum Hance), rooibos tea, rhubarb, pu-erh tea, green tea, “Ougon” (a root of Scutellaria baicalensis Georgi), St. John's wort (Hypericum perforatum L.), licorice, “Senrikou” (Senecio scandens Buch.-Ham.), wintergreen, “Kashi” (a matured fruit of Terminalia chebula Retz.), “Yagotou” (a bark of Mallotus japonicus), polygnum root, barrenwort (Epimedium Herb), guarana, “Ouhi” (a bark of Prunus jamasakura Sieb. ex. Koidz, or a closely related plant thereof), Argy's wormwood, sticky rehmannia, Japanese cornel, Asiasarum root, cinnamon, peony root, pine needle, and amla fruit.

2. A therapeutic and/or prophylactic agent of sex hormone-dependent diseases which comprises the aromatase inhibitor according to claim 1.

3. A method for prophylaxis or treatment of breast cancer of female after menopause or sex hormone-dependent diseases which comprises administering an extract of one or more crude drugs selected from the group consisting of “Kokeiten” (golden root), prunella spike, sweet hydrangea leaf, milk thistle, a jasmine tea, “Bokusoku” (a bark of Quercus acutissima or a closely related plant thereof), “Tencha” (sweet tea), “Karensou” (Eclipta prostrata), “Youbaihi” (a bark of Myrica rubra Sie b. et Zucc.), French maritime pine, betal palm, asparagus, “Rouro” (a root of Rhaponticum unifloru m DC. or Echinops latifolius Tausch), “Ryoukyou” (a rhizome of Alpinia officinarum Hance), rooibos tea, rhubarb, pu-erh tea, green tea, “Ougon” (a root of Scutellaria baicalensis Georgi), St. John's wort (Hypericum perforatum L.), licorice, “Senrikou” (Senecio scandens Buch.-Ham.), wintergreen, “Kashi” (a matured fruit of Terminalia chebula Retz.), “Yagotou” (a bark of Mallotus japonicus), polygnum root, barrenwort (Epimedium Herb), guarana, “Ouhi” (a bark of Prunus jamasakura Sieb. ex. Koidz, or a closely related plant thereof), Argy's wormwood, sticky rehmannia, Japanese cornel, Asiasarum root, cinnamon, peony root, pine needle, and amla fruit to a patient in an amount effective for prophylaxis or treatment of the diseases.

4. The method according to claim 3, wherein “Kokeiten” (golden root) is a material in which a whole petal of Kokeiten (golden root) (Rhodiola sacra Fu) belonging to an orpine family (Crassulaceae) or an underground portion of the other plants belonging to the same genus is dried.

5. The method according to claim 3, wherein prunella spike is a material in which a fruit-spike of self-heal (Prunella vulgaris L. var. lilacina Nak.) belonging to a perilla family (Labiaceae) is dried.

6. The method according to claim 3, wherein sweet hydrangea leaf (hortensia) is a material in which young leaves of hortensia (Hydrangea macrophylla var. thunbergii) which is a variant of a low deciduous tree Hydrangea macrophylla Ser. F. normalis belonging to a saxifrage family (Saxifragaceae) are steamed, rubbed and dried.

7. The method according to claim 3, wherein milk thistle is a material in which a dry fruit of milk thistle (Silybum marianum L., another name: Carduus marianus L.) belonging to a chrysanthemum family (Asteraceae) is dried.

8. The method according to claim 3, wherein the jasmine tea is a material in which green tea and a flower petal of jasmine (Jasminum sambac. (L.) Ait.) are mixed and dried.

9. The method according to claim 3, wherein “Bokusoku” (a bark of Quercus acutissima or a closely related plant thereof) is a material in which a bark of a kind of oak (Quercus acutissima) belonging to a beech family (Fagaceae) or other analogue plants is dried.

10. The method according to claim 3, wherein “Tencha” (sweet tea) is a material in which young leaves of “Rouren Shukyu” (Hydrangea strigosa Rehd.) or Yakushima hydrangea (Hydrangea umbellate Rehd.) belonging to a saxifrage family (Saxifragaceae) are dried.

11. The method according to claim 3, wherein “Karensou” (Eclipta prostrata) is a material in which whole plant of “Takasaburo” (Eclipta prostrata) belonging to a chrysanthemum family (Compositae) is dried.

12. The method according to claim 3, wherein “Youbaihi” (a bark of Myrica rubra Sie b. et Zucc.) is a material in which a bark of bayberry (Myrica rubra Sieb. et Zucc.) belonging to a bayberry family (Myricaceae) is dried.

13. The method according to claim 3, wherein French maritime pine is a material in which a bark of Pinus pinaster or P. maritima belonging to a pine family (Pinaceae) is dried.

14. The method according to claim 3, wherein the sex hormone-dependent disease is selected from male menopausal disorders and metabolic syndrome due to accumulation of fats on internal organs.
